(19) United States
(12) Patent Application Publication
(10) Pub. No.: US 2007/0238997 A1
(43) Pub. Date: Oct. 11, 2007

(54) Ultrasound and fluorescence imaging

(76) Inventor: Estelle Camus, Erlangen (DE)

Correspondence Address: BRINKS HOFER GILSON & LIONE, P.O. BOX 10395, CHICAGO, IL, 60610, US

(21) Appl. No.: 11/393,552

(22) Filed: Mar. 29, 2006

(51) Int. Cl. A61B 8/00; A61B 6/00

(52) U.S. Cl. 600/437; 600/476

(57) ABSTRACT

Medical diagnostic imaging uses ultrasound and light information. Data from both types of imaging are combined, and an image is generated as a function of the combined data. Both types of data are acquired and a three-dimensional representation is generated as a function of one or both types of data. Data from both types of images may be used for quantification. A system may allow selection of use of either one or both types of imaging. The different types of data may be acquired based on a same event in a physiological cycle, such as the P-wave of the heart cycle.

### BACKGROUND

The present embodiments relate to ultrasound and fluorescence imaging. In particular, ultrasound and fluorescence provide medical diagnostic imaging.

Fluorescence (e.g., luminance) imaging and ultrasound imaging (i.e., echography) are both used in characterizing tumors or cancer. For example, these two types of imaging are used in dermatology. Fluorescence imaging offers poor penetration depth and furnishes information only about the surface of the tumors. For example, the light scan penetrates less than a centimeter, such as on the order of magnitude of 500 μm. Ultrasound imaging offers adequate penetration depth (e.g., several millimeters or centimeters), but offers poor resolution at the surface. Neither of these types of imaging offers all the information that may be desired for diagnosis and treatment.

Typically, only one or the other of these two types of imaging is used for diagnosis and treatment. Separate scans from both types of imaging have been used for a same diagnosis. However, it may be difficult for the physician to understand the local conditions of both sets of data. Moreover, both methods are used from time to time by different physicians and/or at different times, possibly resulting in different tissue status for the different scans.

### BRIEF SUMMARY

By way of introduction, the preferred embodiments described below include methods, systems and computer readable media for medical diagnostic imaging using ultrasound and light information. Data from both types of imaging are combined, and an image is generated as a function of the combined data. Both types of data are acquired and a three-dimensional representation is generated as a function of one or both types of data. Data from both types of images may be used for quantification. A system may allow selection of use of either one or both types of imaging. The different types of data may be acquired based on a same event in a physiological cycle, such as the P-wave of the heart cycle. Any one or combination of two or more of the features above may be used.

In a first aspect, a method is provided for medical diagnostic imaging. Ultrasound data representing at least a first region is acquired. Fluorescence data representing at least the first region is acquired. The ultrasound data and the fluorescence data representing the first region are combined. An image is generated as a function of the combined ultrasound and fluorescence data.

In a second aspect, a system is provided for medical diagnostic imaging. An ultrasound data path is operable to acquire ultrasound data representing at least a first region. A light scan data path is operable to acquire light data representing at least the first region. A user input is operable to receive selection of an ultrasound scan, a fluorescence scan or combinations thereof. A processor is operable to generate an image as a function of the ultrasound data, the fluorescence data, or combinations thereof. The processor is responsive to the selection.

In a third aspect, a computer readable storage medium has stored therein data representing instructions executable by a programmed processor for medical diagnostic imaging. The instructions are for obtaining first data responsive to ultrasound energy and second data responsive to light energy, and generating a three-dimensional representation as a function of both the first and second data.

In a fourth aspect, a method is provided for medical diagnostic imaging. Ultrasound data and fluorescence data representing at least the first region are acquired. A three-dimensional representation is generated as a function of the ultrasound and fluorescence data.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for medical diagnostic ultrasound imaging;

FIG. 2 is a graphical representation of combination of data from ultrasound and light scans according to one embodiment; and

FIG. 3 is a block diagram of one embodiment of a system for medical diagnostic ultrasound and fluorescence imaging.

### DETAILED DESCRIPTION OF THE DRAWINGS AND PRESENTLY PREFERRED EMBODIMENTS

The fluorescence and ultrasound imaging are combined for characterizing tumors. The two types of imaging are used for quantification, data combination and/or three-dimensional visualization. For example, an ultrasound data set is obtained. A fluorescence data set is obtained. Both data sets are recorded and used for two- or three-dimensional visualization or quantitative analysis. Information about the tissue type is provided for the surface and at depth, allowing volume calculation and three-dimensional imaging. Such information may assist in dermatology diagnosis, in surgery or other applications.

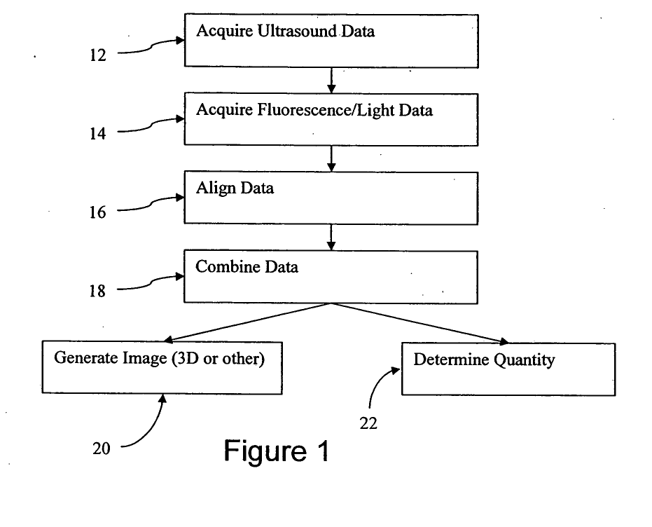

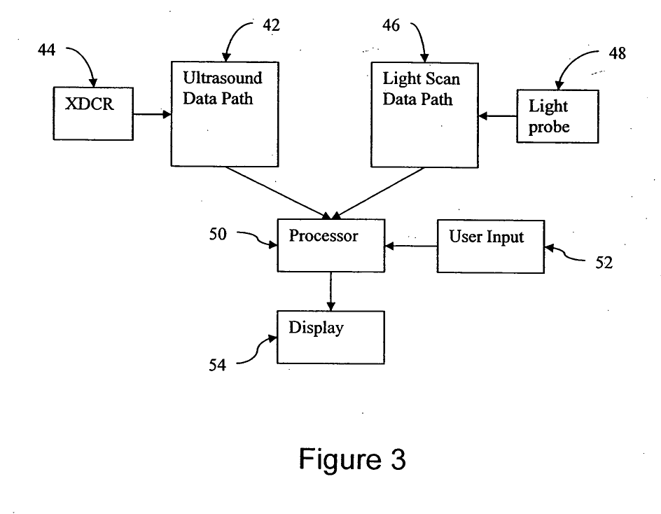

FIG. 1 shows a method for medical diagnostic imaging. The method is implemented by the system of FIG. 3 or a different system. The method is performed in the order shown or a different order, such as performing act 14 before act 12. Additional, different or fewer acts may be provided, such as performing one of acts 18, 20 or 22 without one or more of the other acts.

In act 12, ultrasound data representing at least a first region is acquired. The ultrasound data is acquired by scanning with acoustic energy. Alternatively, the ultrasound data is acquired from a database or memory, such as loading the ultrasound data from an archival (e.g., PACs) system.

The ultrasound data represents one or more spatial locations, such as a two-dimensional region. Any scan format may be used, such as sector, Vector®, or linear. In one embodiment, the ultrasound data represents a volume or three-dimensional region. A three-dimensional scan using a two-dimensional array or a plurality of spatially offset two-dimensional scans may be used to acquire the ultrasound data. The ultrasound data along one or more scan lines extending into a patient or away from a transducer provide information about structures, tissue or fluid spaced from the transducer. For example, a three-dimensional scan results in information about structures deep (e.g., centimeters) in tissue. Information associated with shallower depths may be acquired.

Any now known or later developed ultrasound scanning may be used. For example, B-mode, Doppler flow, Doppler tissue, spectral Doppler, contrast agent, harmonic, or tissue harmonic pulse sequences and/or detection techniques may be used. The ultrasound data is intensity, velocity, frequency shift, energy, power, variance or other characteristic of signals reflected from tissue, boundaries, fluid, or contrast agents. The ultrasound data may be derived from reflected echoes, such as beamformed, detected, filtered, scan converted or at another stage in an ultrasound image process.

In act 14, fluorescence data representing at least the first region is acquired. The fluorescence data is acquired by light scanning. Alternatively, the fluorescence data is acquired from a database or memory, such as loading the fluorescence data from an archival (e.g., PACs) system.

Fluorescence data is data responsive to light wavelengths and include infrared detection. Fluorescence is excited, such as by light excitation. The excitation light is in the visible or in the infrared range (IR), for example, or in the near infrared range (NIR). The suitable frequency range is dependent on the substance to be examined. Other light wavelengths may be used. A region marked fluorescently is exposed to light in at least the desired excitation wavelength of the fluorescent dye. For detection, a detector is capable of detecting light in the wavelength that the substance emits or allows to pass-through upon excitation.

Fluorescence detection may detect various molecular factors. Substances having different molecular properties may have different fluorescent properties, which can be detected in a targeted way. Fluorescence detection is optically based and is noninvasive or minimally invasive. With the knowledge of the applicable fluorescent properties, the molecular nature of a given material being examined may be ascertained.

In medicine, molecular properties, for instance also called a “molecular signature,” provide information about the state of health of a living creature or patient and can be assessed diagnostically. For example, molecular signatures are used for detecting cancer. Still other syndromes, such as rheumatoid arthritis or arteriosclerosis of the carotid artery, can be identified.

Substances that have no molecular or chemical properties that would be suitable for fluorescence detection can be molecularly “marked” in a suitable way. For example, suitably prepared markers bind to or are deposited only on particular molecules. The marker and the molecule to be detected fit one another while the marker does not bind to other substances. If the marker has known fluorescent properties, then the marker may be optically detected after binding. The detection of the marker allows conclusions to be drawn as to the presence of the marked substance.

Fluorescent metabolic markers accumulate only in certain regions, such as tumors, infections, or other foci of disease, or are distributed throughout the body. Such markers may be activated only in certain regions, for instance by tumor-specific enzyme activities with additional exposure to light. In medical diagnosis, marker substances, so-called fluorophores, such as indocyanin green (ICG), bind to blood vessels and can be detected optically. In an imaging process, the contrast with which blood vessels may be enhanced. So-called “smart contrast agents” may be used. These are activatable fluorescence markers bind, for instance, to tumor tissue. The fluorescent properties are not activated until the binding to the substance to be marked occurs. Such substances may comprise self-quenched dyes, such as Cy5.5, which are bound to larger molecules by way of specific peptides. The peptides can in turn be detected by specific proteases, produced for instance in tumors, and can be cleaved. The fluorophores are released by the cleavage and are no longer self-quenched, but instead develop fluorescent properties. The released fluorophores can be activated for instance in the near IR wavelength range of around 740 nm. One example of a marker on this basis is AF 750 (Alexa Fluor 750), with a defined absorption and emission spectrum in the wavelength range of 750 nm (excitation) and 780 nm (emission).

In medical diagnosis, such activatable markers may be used, for instance, for intraoperative detection of tumor tissue. The diseased tissue may be identified exactly and then removed. One typical application is the surgical treatment of ovarian cancer. The diseased tissue is typically removed surgically. Because of the increased sensitivity of fluorescence detection, the diseased tissue can be better detected along with various surrounding foci of disease and thus removed more completely.

In the treatment of breast cancer, typical surgical treatments are lumpectomies or mastectomies and lymph node sections and biopsies. Lymph nodes are typically detected optically by means of 99mTc sulfur colloids in combination with low-molecular methylene blue. The radioactive mTc sulfur colloids could be avoided by using fluorescence detection.

In the removal of brain tumors, the precise demarcation of the tumor tissue, which is attainable by the use of fluorescence detection, is important. The treatment of pancreatic tumors can benefit from additional lymph node biopsies, which could be identified by fluorescence detection, to detect possible intestinal cancer. In the treatment of skin cancer, the detection of skin neoplasms may be improved by fluorescence detection. Fluorescence detection may improve medication monitoring, such as for rheumatoid arthritic diseases of joints. The extent of protease overproduction may be detected quantitatively, and the medication provided to counteract overproduction may be adapted quantitatively.

In treating these diseases named as examples as well as other syndromes, an operation is typically performed to remove surgically diseased tissue. For aiding in the operation, fluorescence detection may be performed to improve the detection of the diseased tissue portions to be removed during the ongoing operation or in the open wound. To that end, the tissue parts are marked before the operation with a suitable substance that is then activated by binding to the diseased tissue parts.

Fluorescence methods examine regions near the surface or in the open body (e.g., intraoperative applications). Examples of such investigations are detecting fluorescently marked skin cancer or the detection of tumor boundaries in the resection of fluorescently marked tumors. For example, coronary arteries and the function of bypasses (that is, the flow through them) are intraoperatively viewed. The scanned region is generally two-dimensional, but may be a point, a plurality of points, a three-dimensional surface or a three-dimensional volume. For example, the scan is of a generally flat two-dimensional surface (e.g., skin) or a three-dimensional surface (e.g., outer surface of an internal organ). As another example, the light penetrates to a plurality of depths, allowing for a volume scan.

Other information may be included with the fluorescence data, such as optical data based on visible light. The optical and the fluorescence information are superimposed to indicate a context of the fluorescence data. If a scan based on fluorescence is generated by light in the IR or NIR range, then the user may not see the excitation light. Visual light markers or beams may be used with or without detection of visible information. For example, visual light optical images are generated on an ongoing basis so that the surgeon can observe the images in real time. The surgeon can aim the scanner based on the images. Once aimed, the fluorescence scanner is activated to acquire fluorescence data.

Acts 12 and/or 14 are performed as a function of a physiological event. For example, scans for acquiring are triggered at a particular time in a cycle. As another example, data previously acquired at a particular time is selected from other data.

Both the ultrasound and fluorescence data are associated with a same physiological event of a cycle. For example, both types of data are acquired at p-wave or other portions of a heart cycle. The same or different cycle may be used for the different types of data, such as acquiring the fluorescence data at the p-wave of one cycle and the ultrasound data at the p-wave of another cycle. If the motion of the heart or respiration influences the data, physiological parameters such as EKG or the motion of the thorax are used for triggering acquisition or gating previously acquired data. The data is selected or triggered to belong to the same physiological phase.

In act 16, the ultrasound and fluorescence data are aligned. The alignment is performed prior to combining the data for imaging or quantification. The alignment may be used for imaging even where the different types of data are not combined, such as for side-by-side presentation of different types of images.

The data is aligned spatially by shifting, rotating, modifying, morphing or transforming the data. The spatial locations represented by one type of data are aligned to correspond to the same spatial locations represented by another type of data. The alignment is with reference to the scanned region or patient. Ultrasound data representing a feature is aligned with fluorescence data representing a same feature rather than a different feature.

The relative positions for alignment are determined from one or more position sensors, from the data or combinations thereof. Magnetic, motion, ultrasound, optical, or other position sensors determine the position and/or orientation of a probe and associated data relative to a defined frame of reference. The probes for both the ultrasound and fluorescence scans include position sensors, or a single probe for both types of scans includes a position sensor. The different types of data may be acquired at different times, but the relative position is known from the position sensors. During the scans, the positions of the applicators are determined. The known or measured position of the probes relative to the position sensors and/or transducer or light sensors provides an indication of position of the scan region represented by the data. Alternatively, the different types of data are acquired at a same time with a known or measured spatial relationship between the two probes or scan devices.

Alternatively or additionally, data representing anatomical and/or artificial landmarks are identified in each of the different types of data. For example, a tissue structure or marker on the surface of the scanned tissue is visible by both fluorescence and ultrasound methods. These features or landmarks are aligned to align the data. Correlation of features from one type of data to features of another type of data is performed. Different relative alignments are attempted to identify the alignment with the highest or sufficient correlation. Any correlation function may be used, such as cross-correlation or sum of differences.

In act 18, the ultrasound data and the fluorescence data are combined. The data representing the same locations is combined. For example, the ultrasound data and the fluorescence data both represent a two- or three-dimensional surface in common. Data representing non-overlapping locations is discarded or maintained as part of the combined data set. For example, a set of data representing a three-dimensional volume is provided with a portion of the data being from a combination of the different types of data.

The combination is averaging, compounding, weighted averaging, combinations thereof, or other now known or later developed compounding. For example, a finite impulse response combination with two taps, one for each type of data, is used. The coefficients or weights for each tap determine the relative contribution of the different types of data to the combined data value. Equal or unequal weighting may be used, such as providing a greater weight for fluorescence data closer to or on a surface and a greater weight for ultrasound data deeper in the scanned tissue.

In one embodiment, the ultrasound and fluorescence data are acquired with a similar or same resolution. The different types of data are more readily combined without generating artifacts. In other embodiments, one or both types of data are processed to provide a similar resolution, such as transforming or spatially filtering and decimating the data with a higher resolution. In another embodiment, the data with the lower resolution is up-sampled or interpolated for combination with higher resolution data.

In act 20, an image is generated as a function of the combined ultrasound and fluorescence data. In one embodiment, the combined data represents a line or two-dimensional region. A two-dimensional image is generated.

In another embodiment, a three-dimensional representation is generated. At least a portion of the representation is a function of the combined ultrasound and fluorescence data. Other portions may be from uncombined data, such as ultrasound data. For example, the contribution of a two-dimensional region for the three-dimensional representation is a function of both ultrasound and fluorescence data, but contributions for other locations in the volume is from a single type of data.

Any three-dimensional rendering may be used, such as minimum, maximum, average or other projection rendering or surface rendering. In projection rendering, a plurality of parallel or diverging ray or view lines is projected through the volume. One ray line is provided for each display pixel of the representation. Data for spatial locations along or adjacent each line is used to determine a value for the ray line. For example, a first value along the ray line that is over a threshold is selected. A three-dimensional representation is generated from the selected values. Where one or more ray lines pass through locations represented by combined data, the three-dimensional representation is, at least in part, a function of the combined data even if the combined data value is not selected or averaged to determine the pixel value.

Opacity weighting or other three-dimensional rendering process may be used. The data set for rendering is transformed to a three-dimensional grid for rendering, but may be free of transformation. Combination may occur before or after any transformation. The pixel values are black and white or color values.

In another embodiment, a three-dimensional representation is generated as a function of the ultrasound and fluorescence data without combining the data. For example, a set of data representing a volume includes data for some locations from fluorescence data and data for other locations from ultrasound data. A three-dimensional representation is rendered from the data set. Alternatively, the data set includes separate data from the different types of data and some combined data.

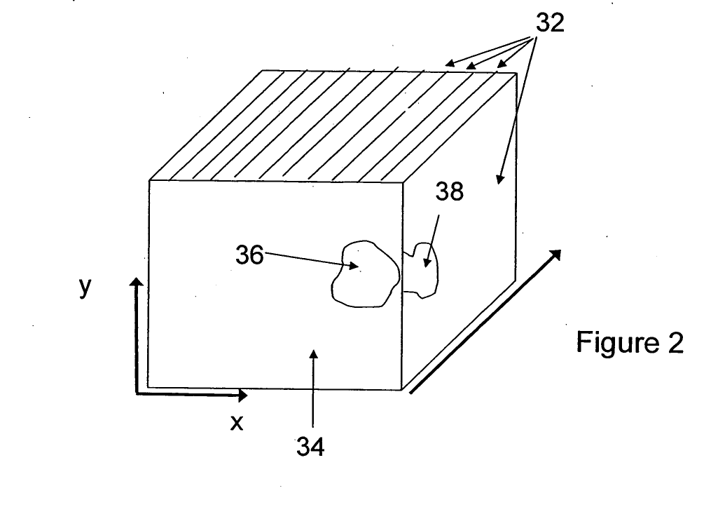

FIG. 2 shows one embodiment of a data set representing a volume. A plurality of two-dimensional planes 32 of ultrasound data are provided with one two-dimensional plane 34 of fluorescence data. The fluorescence plane 34 is generally perpendicular to the planes 32 of ultrasound data. The fluorescence plane 34 represents a skin or other tissue surface. The fluorescence data shows a feature 36, such as a tumor. Other characteristics of the feature are shown by the ultrasound data at 38. By combining or using both types of data, information about the entire tumor may more likely be provided. Using both types of data, different diagnostic information may be provided, such as tissue type or tumor identification using fluorescence data and spatial characteristic from both types of data.

In act 22 of FIG. 1, a quantity is determined as a function of the ultrasound and fluorescence data. The quantity is calculated from combined data or both types of data maintained separately. For example, the data set representing the volume is used to derive contours or borders. Automatic processes, such as thresholding or region growing, identify the borders. Alternatively or additionally, the user manually enters one or more indications of the border location from a displayed image. The borders are used to determine a volume, circumference, length, width or other characteristic. Other quantities may be calculated, such as the volume flow through the region.

FIG. 3 shows a system for medical diagnostic imaging. The system includes an ultrasound data path 42, a transducer 44, a light scan data path 46, a light probe 48, a processor 50, a user input 52 and a display 54. Additional, different or fewer components may be provided, such as not including the transducer 44 or the light probe 48.

The ultrasound data path 42 includes a beamformer, detector, scan converter, and/or other ultrasound imaging system component. The ultrasound data path is operable to acquire ultrasound data representing at least a first region. Using the one or two-dimensional array of elements on the transducer 44, the ultrasound data path scans with ultrasound energy and generates ultrasound data as a function of the scanning. For example, the transducer 44 operates with transmit and receive beamformers. The output of the receive beamformer is provided on a B-mode path, a color Doppler path and/or a spectral Doppler path. Detectors detect the intensity, velocity, energy, velocity range, turbulence or other characteristic for a location, two-dimensional area or a three dimensional volume. An optional scan converter formats the detected information as an image for the display 54. Additional, different or fewer components may be provided, such as including filters and/or image processors. Other ultrasound imaging systems may be used.

The light scan data path 46 is operable to acquire light data representing at least the first region. In one embodiment, the light scan data path 46 is a fluorescence scanner. The fluorescence scanner connects with the light probe 48. A surgeon or other user manipulates the light probe 48 to a desired position. A button may be used to trigger manually a fluorescence scan. A plurality of excitation light sources is arranged on the light probe 48 to illuminate a region at a distance of approximately 6 to 10 cm. The light sources are arranged at an angle of approximately 45° or other angle to a front panel of the light probe 48. This arrangement may provide an optimal working distance since the scanning region should not be touched and too great a distance would require excessively high excitation light intensity. Other distances and/or angles may be used.

The excitation light sources may be halogen illuminants, but may be LEDs (light emitting diodes). Since an individual LED has a relatively low luminous intensity, LED arrays about magnitude of 60 LEDs each may be used for each light source. For example, each of four LED arrays has a total luminous power of approximately 0.25 to 1 Watt.

A lens on the fluorescent scanner is aimed frontally at the illuminated region. Fluorescent light, normal light and ambient light may reach the fluorescence scanner through the lens. So that the fluorescent light will not be washed out by the ambient light, a filter allows fluorescent light, but reduces or removes light in the visible wavelength ranges. To enable making an optical image in the visible wavelength range, a filter changer can for instance change to a filter that allows light in the visible wavelength range to pass through. Light passed through the lens and the filter reaches a CCD camera. The CCD camera records images of signals at the desired wavelength range (e.g., visible light and/or in the wavelength range of the fluorescence). The image data recorded by the CCD camera is received by a data acquisition unit and stored or transmitted. In one embodiment, the fluorescence scanner operates on reflected light. In other embodiments, the camera of the fluorescence scanner is positioned to detect light transmitted through tissue.

In one embodiment, the ultrasound data path 42 is operated to image the tissue of interest. Once the tissue of interest is identified, a fluorescence scan is made. The fluorescence scan is made from a probe 48 in a particular spatial relationship to the ultrasound transducer 22 so that the location found with ultrasound imaging may be used to aim the light probe 48. Alternatively, the fluorescence data path 46 includes mechanisms, such as visual imaging or a target beam projection, to aim the light probe 48.

In one embodiment, the light probe 48 and the transducer 44 are provided in a same device or housing. For example, transducer elements and camera elements are provided on a same surface with or without light sources. The light probe 48 and transducer 44 are positioned to scan a same or overlapping region with a known spatial relationship. Alternatively, separate housings are provided for the light probe 48 and the transducer 44.

In one embodiment, the ultrasound data path 42 and the light scan data path 46 are provided in a same device, such as in a same housing, on a same cart, or connected together locally. The processor 50, user input 52, and display 54 are part of the same device or are separate, such as being a workstation (e.g., PACs) or other computer. Alternatively, the ultrasound data path 42 and the light scan data path 46 are separate devices or imaging systems.

The user input 52 is a keyboard, mouse, trackball, button, slider, touch pad, touch screen, knob, combinations thereof, or other now known or later developed user input device. The user input 52 accepts input from the user for controlling the processor 50, the ultrasound data path 42, and/or the light scan data path 46. In one embodiment, the user input 52 is a user input of the ultrasound data path 42, and/or the light scan data path 46. The user input 52 is operable to receive selection of an ultrasound scan, a fluorescence scan or combinations thereof. For example, the user initiates or controls the fluorescence scan and the ultrasound scan from a same user interface. Alternatively, separate user interfaces and/or user inputs are provided.

The processor 50 is a general processor, control processor, digital signal processor, server, application specific integrated circuit, field programmable gate array, digital circuit, analog circuit, combinations thereof or other now known or later developed device for controlling acquisition and/or processing data. The processor 50 is a single device or a plurality of devices, such as a network or parallel processors. In one embodiment, the processor 50 is a processor of the ultrasound data path 42 or the light scan data path 46. In other embodiments, the processor 50 is separate, but operable to receive data from both data paths 42, 46. With a combined unit, the applications for fluorescence imaging and ultrasound imaging are controlled from the same processor 50, and the data sets are stored in memory jointly. The visualization or quantification is performed by the processor 50 or another platform.

The processor 50 is operable to implement the method of FIG. 1 or a different method. In one embodiment, the processor 50 identifies ultrasound data and fluorescence data for processing. For example, the different types of data associated with a same physiological event or phase of a cycle are obtained. The data is obtained by triggering scans as a function of sensed trigger events or by selecting stored data associated with the desired phase of the cycle.

The processor 50 is operable to combine the ultrasound and fluorescence data representing a same or overlapping region. Values for two different types of data representing a same location, such as pixel display values, are added, subtracted, averaged or otherwise combined. Alternatively, a data set is created from the two types of data without combining values.

The processor 50 is operable to generate an image as a function of the ultrasound data, the fluorescence data, or combinations thereof. The combination of the two types of data is either combined data, such as averaged values, or a data set with each value being responsive to only one of the two types of data. The processor 50 generates a two-dimensional image or three-dimensional representation. Alternatively or additionally, the processor 50 determines a quantity as a function of the ultrasound and fluorescence data.

In one embodiment, the processor 50 operates substantially continuously or in real-time to scan the patient. Generated images are used to view and assist in navigation of a biopsy needle.

The display 54 is a CRT, LCD, plasma, projector, printer or other now known or later developed display device. The display 54 connects with the processor 50, but may alternatively connect to the ultrasound data path 42 or through the light scan data path 46. The display 54 is part of, rests on, adjacent to or remote from a housing for the processor 50.

The instructions for controlling the processor 50 are stored in a computer readable media. The computer readable storage medium has stored therein data representing instructions executable by a programmed processor for medical diagnostic imaging. The instructions are for obtaining data responsive to ultrasound energy and data responsive to light energy. The data associated with a same physiological event of a cycle is identified. The data is used for combination, generating a two- or three-dimensional representation and/or determining a quantity. In one embodiment, the instructions are for generating a three-dimensional representation as a function of the combined data for at least one location. Other processes may be implemented by the computer instructions.

The instructions for implementing the processes, methods and/or techniques discussed above are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I(We) claim:

1. A method for medical diagnostic imaging, the method comprising:
acquiring ultrasound data representing at least a first region;
acquiring fluorescence data representing at least the first region;
combining the ultrasound data and the fluorescence data representing the first region; and
generating an image as a function of the combined ultrasound and fluorescence data.

2. The method of claim 1 wherein generating the image comprises generating a three-dimensional representation.

3. The method of claim 2 wherein the first region comprises a two-dimensional region, wherein acquiring the ultrasound data comprises acquiring the ultrasound data representing a volume including the two-dimensional region, wherein acquiring the fluorescence data comprises acquiring the fluorescence data representing the two-dimensional region, wherein combining comprises combining the ultrasound and fluorescence data for the two-dimensional region, and wherein generating comprises generating with the contribution of the two-dimensional region for the three-dimensional representation being a function of both ultrasound and fluorescence data.

4. The method of claim 1 wherein combining comprises averaging, compounding, weighted averaging or combinations thereof.

5. The method of claim 1 wherein acquiring the ultrasound and fluorescence data comprises acquiring as a function of a physiological event.

6. The method of claim 1 further comprising:
determining a quantity as a function of the ultrasound and fluorescence data.

7. The method of claim 1 further comprising:
aligning the ultrasound and fluorescence data prior to combining, the alignment being a function of a position sensor, features represented by the ultrasound and fluorescence data, or combinations thereof.

8. A system for medical diagnostic imaging, the system comprising:
an ultrasound data path operable to acquire ultrasound data representing at least a first region;
a light scan data path operable to acquire light data representing at least the first region;
a user input operable to receive selection of an ultrasound scan, a fluorescence scan or combinations thereof; and
a processor operable to generate an image as a function of the ultrasound data, the fluorescence data, or combinations thereof, the processor being responsive to the selection.

9. The system of claim 8 wherein the processor is operable to combine the ultrasound data and the fluorescence data representing the first region.

10. The system of claim 9 wherein the processor is operable to align the ultrasound and fluorescence data prior to combination.

11. The system of claim 8 wherein the processor is operable to generate a three-dimensional representation.

12. The system of claim 8 wherein the processor is operable to identify the ultrasound data and the fluorescence data associated with a physiological event of a cycle.

13. The system of claim 8 wherein the processor is operable to determine a quantity as a function of the ultrasound and fluorescence data.

14. In a computer readable storage medium having stored therein data representing instructions executable by a programmed processor for medical diagnostic imaging, the storage medium comprising instructions for:
obtaining first data responsive to ultrasound energy;
obtaining second data responsive to light energy;
generating a three-dimensional representation as a function of both the first and second data.

15. The instructions of claim 14 further comprising:
combining the first and second data;
wherein the three-dimensional representation is a function of the combined data for at least one location.

16. The instructions of claim 14 further comprising:
identifying the first and second data associated with a same physiological event of a cycle.

17. The instructions of claim 14 further comprising:
determining a quantity as a function of the ultrasound and fluorescence data.

18. The instructions of claim 14 further comprising:
aligning the first and second data prior to generating the three-dimensional representation.

19. A method for medical diagnostic imaging, the method comprising:
acquiring ultrasound data representing at least a first region;
acquiring fluorescence data representing at least the first region; and
generating a three-dimensional representation as a function of the ultrasound and fluorescence data.

20. The method of claim 19 further comprising:
combining the ultrasound data and the fluorescence data representing the first region;
wherein generating the three-dimensional representation comprises generating as a function of the combined data.

21. The method of claim 20 further comprising:
aligning the ultrasound and fluorescence data prior to combining.

22. The method of claim 19 further comprising:
identifying the ultrasound data and fluorescence data associated with a same physiological event of a cycle.

23. The method of claim 19 further comprising:
determining a quantity as a function of the ultrasound and fluorescence data.
